(19) United States
(12) Patent Application Publication
(10) Pub. No.: US 2015/0039038 A1
(43) Pub. Date: Feb. 5, 2015

(54) Orthopedic Screw Fastener System Including Locking and Non-Locking Screws

(71) Applicants: Stephan Eckhof, Weilheim-Rietheim (DE); Josef Zrinski, Wurmlingen (DE); Zoran Reiter, Emmeningen-Liptingen (DE); Frank Altermann, Tuttlingen (DE); Rolf Maier, Liptingen (DE); Davor Zrinski, Weilheim-Rietheim (DE); Oliver Burckhardt, Philadelphia, PA (US)

(72) Inventors: Stephan Eckhof, Weilheim-Rietheim (DE); Josef Zrinski, Wurmlingen (DE); Zoran Reiter, Emmeningen-Liptingen (DE); Frank Altermann, Tuttlingen (DE); Rolf Maier, Liptingen (DE); Davor Zrinski, Weilheim-Rietheim (DE); Oliver Burckhardt, Philadelphia, PA (US)

(21) Appl. No.: 13/959,093

(22) Filed: Aug. 5, 2013

(51) Int. Cl. A61B 17/80

(52) U.S. Cl. CPC A61B 17/8057
USPC 606/291

(57) ABSTRACT

An orthopedic screw fastener system which includes at least two screws and at least one bone plate. At least one screw is a locking screw comprising a screw head and a screw shank, the screw head being provided with a thread. At least one other screw is a non-locking screw comprising a non-threaded (i.e., smooth) screw head and a screw shank. The at least one bone plate has a thickness and it has at least two through-bores which are able to receive screws. The plate has two or more through-openings, each formed by at least two through-bores, each through-bore being defined by a midpoint and by a radius, and the through-bores are offset relative to each other and intersect each other in such a way that intersection lines and/or intersection surfaces extend into the depth of the through-opening.

### BACKGROUND TO THE INVENTION

1. Field of the Invention

The present invention relates to a screw fastener system of the type described in our co-pending U.S. patent application Ser. No. 12/002,625, filed Dec. 18, 2007, the disclosure of which is incorporated herein by reference. The invention comprises at least two screws and at least one bone plate with at least two through-bores. At least one screw consists of a screw head and a screw shank, the screw head being provided with a locking thread to lock the screw in the bone plate, hereinafter referred to as a “locking screw”. At least one other screw consists of a screw head and a screw shank, but the screw head is not threaded and it is non-locking when screwed into a bone plate, hereinafter referred to as a “non-locking screw”. Both types of screws may be referred to herein occasionally as a “screw” or “screws”. The screws can be inserted multiaxially into the through-bores. This means that the screws can be inserted, not perpendicular to the plate, but obliquely with respect to the plate, and at least one can be locked on the plate while at least one is not locked on the plate. A force-fit connection, or a force-fit and form-fit connection, is obtained between the locking screw and the plate.

### DEFINITIONS

### Through-Bore

Through-bore defines bores that extend from the top of a plate to the underside of the plate. It can be cylindrical or conical in shape. Generally, it can be produced using a drill or a milling cutter.

### Through-Opening

Through-opening defines openings that are suitable for receiving a screw with screw shank and screw head. In the through-opening, the locking screw head establishes the force-fit and form-fit connection with the rest of the plate. The through-opening is generally formed initially by two or more through-bores that are arranged relative to one another in such a way that they intersect. To produce a through-opening, a central bore is first made, and further through-bores are formed from this at regular intervals, such that a structure is obtained which, in plan view, looks like a clover or flower or leaf. A non-locking screw can be screwed into the same through-opening without locking because the screw head of the non-locking screw is not threaded.

### Intersection Line

If several through-bores intersect, then intersection lines form in the area of the through-openings and extend through the thickness of the plate. The intersection lines thus extend into the depth of the through-opening. They are preferably configured such that they extend in the direction of the longitudinal extent of the through-bore. The intersection lines are elements that interact with the locking screw head to bring about a force-fit and form-fit connection through plastic deformation.

### Intersection Surface

The intersection lines defined above then become intersection surfaces, if the through-bore and/or the central bore are conical in shape. The intersection surfaces are elements that interact with the locking screw head to bring about a force-fit and form-fit connection through plastic deformation. For simplicity, only intersection lines will be discussed below. Unless otherwise stated, however, this is also understood to cover the combination of intersection lines and intersection surfaces or only the intersection surfaces.

2. The Prior Art

Screws and plates, in particular bone plates, of the aforementioned type generally form a fixation system and are mainly used to mechanically stabilize bone fractures. This fixation system consists of a plate, which is provided with at least one through-bore, but generally with several through-bores, said through-bores being designed in each case to receive a screw. The screw itself consists of a screw head and of a screw shank and, on the screw shank, has a thread that is able to be screwed into a bone. The screw head itself also has a thread which, in the assembled state of the fixation system, interacts with the plate in the respective through-bore.

Orthopedic fixation devices can be used both outside and also inside the body. They consist of a plate-shaped structure, which extends over a fracture, for example. Securing means are also provided, which are designed, for example, as screws, bolts, nails or pins. For example, so-called bone plates can be secured on a bone by means of bone screws, by the latter being inserted through through-bores and being screwed into the bone. To ensure that these screws do not come loose, particularly under the effect of forces, a great many mechanisms are known from the prior art which avoid loosening of the screw and, consequently, loosening of the plate-shaped structure. Solutions are also known that permit, between the plate and the screw, a force-fit connection in which the screw is not oriented perpendicular to the plate. An oblique position of such a screw is often desired if, for example on account of the bone structure, it is not possible for the screw to be screwed perpendicular to the plate.

Thus, for example, so-called expansion-head screws are known from U.S. Pat. No. 4,484,570 (SYNTHES LTD (US)) Nov. 27, 1984. A head screw that has been screwed into a plate is spread open by an additional screw element in such a way that, by screwing in the additional screw, a wedging effect takes place within the plate, such that the screw is fixed on the plate.

Another embodiment from the prior art, as set out for example in U.S. Pat. No. 5,954,722 (DEPUY ACROMED INC (US)) Sep. 21, 1999, comprises a plate into which a screw can be screwed multiaxially. The multiaxial aspect has, among other things, the advantage that the screwing-in directions are no longer defined by the plate itself. In this way, depending on the nature of the material, it is no longer essential for a screw to be screwed into a bone perpendicular to the longitudinal extent of the plate. For this purpose, the through-bores are additionally provided with a spherical element, which is mounted so as to be at least partially rotatable within the through-bore. This spherical element is arranged captive within the through-bore and interacts with the screw head of the screw that is to be screwed in. The spherical element orients itself depending on the position and thus establishes a force-fit and form-fit connection with the screw head and the plate.

Another multiaxial design of a plate, which interacts with a specially designed screw, is set out in US 20050165400 A (FERNANDEZ ALBERTO A, UY) Jan. 26, 2004. The orthopedic fixation system comprises a plate, which likewise has one or more through-bores. A screw is also provided, which consists of a screw head and of a screw shank. The screw head is specially designed and has a thread that interacts with threads provided at least partially in the through-bores.

To allow a screw to be screwed in obliquely (and thus not perpendicular) to the longitudinal extent of the plate, provision is made for the through-bore to be specially designed. The through-bores have an hourglass-shaped cross section. This means that, seen in the screwing-in direction, the diameter of the through-bore narrows from an initially wide diameter until a defined plane is reached. Starting from this plane, the diameter of the through-bore widens out again.

The through-bore formed in this way has a thread, which interacts with the thread of the screw head. The thread is specially designed and constitutes a so-called cutting thread.

This means that, when the screw head is being screwed in, a mechanical cutting process takes place between said thread and the screw head. To strengthen this cutting process and thereby achieve a wedging of the screw head within the through-bore, such that undesired loosening of plate and screw at a later point is no longer possible, cutting elements preferably made of another material are provided within the through-bore. These cutting elements are let into the circumference of the through-bore and bring about a deformation when the screw is screwed into the through-bore. By means of the spherical shape of the screw head, it is possible to choose a large number of angle degrees that deviate from the line perpendicular to the longitudinal extent of the plate.

DE 202004015912U (AESCULAP AG) Dec. 9, 2004 also discloses a fixation system that consists principally of a plate and of a bone screw. The bone screw itself has a shank, which defines a longitudinal axis, and a screw head, which can be brought into engagement with a bone screw seat. A securing element for securing the connection of bone screw and bone plate is additionally provided, the bone screw being able to be brought from a position of engagement, in which the bone screw is held on the bone plate, to a release position.

The through-bores within the bone plate are preferably oval in shape, thereby permitting multiaxial engagement of the bone screw. On their walls, they have threads that can be brought into engagement with the thread of the bone screw or screw head. Securing of the bone screw is achieved by wedging the bone screw to the bone plate. The through-bores are produced by milling an oval through-opening, which has walls formed perpendicular to the longitudinal extent of the bone plate. The walls additionally have thread turns. The production is therefore expensive and complex. The bone screw itself has to be specially designed and have the special securing mechanism available.

WO 20041084701 A (SWISS ORTHOPEDIC SOLUTIONS SA (CH); YOUNG ROBERT ALLAN (US)) Oct. 7, 2004 discloses a bone plate having a longitudinal extent. The bone plate itself has several through-openings, formed in each case by two through-bores offset relative to each other. The through-bores are arranged in such a way that their midpoint is arranged on the center axis of the respective bone plate and arranged at a defined distance from one another. The two through-bores intersect each other in such a way that, in plan view, a configuration in the shape of an eight is obtained. This means that a narrowing is provided between the two through-bores that form the through-opening. In the lower area, that is to say the area facing toward the bone, both through-bores have thread turns that are different than each other. On the side facing away from the bone, that is to say the top, the area of aperture of the through-bore, and thus also of the through-opening, is much greater than in the lower area.

In particular illustrative embodiments, the thread turns provided in the lower area of the bone plate are arranged at an angle to one another or obliquely.

As in the other prior art, it is necessary to produce the bone plate in different work cycles. In particular, in the lower area of the bone plate, that is to say the area facing toward the bone, it proves difficult to arrange different thread turns that can then interact with the head of the bone screw, which likewise has a thread. A free multiaxial arrangement of the bone screw is therefore not possible, since, when being screwed in, it inevitably interacts with the thread and thus also assumes the predetermined direction.

DE 20321245U (SYNTHES GMBH (CH)) Jun. 14, 2006 also discloses a bone plate with an underside facing toward the bone and with a top, and with several through-bores, which connect the underside to the top and each have a central hole axis, an inner jacket surface and a thread turn. In a particular illustrative embodiment, the through-opening is formed by a central through-bore with, arranged on the circumference of the through-bore, through-bores arranged at an angle distance of in each case 90 degrees from one another.

This permits a multiaxial arrangement of the respective bone screws, the head thereof each being inserted into the respective through-bore. The respective through-bores likewise comprise thread turns, as has already been described above.

For this reason, the production is also very complex, and the multiaxial direction of the respective bone screw cannot be freely chosen, on account of the corresponding specifications of the through-bores.

Moreover, there is no possibility of securing the respective bone screw, such that undesired loosening is possible at any time, as a result of which there is a danger of the function of the respective bone plate being completely lost. In some cases, however, it is desirable to use at least one non-locking screw in combination with at least one locking screw and this is the improvement addressed by the present invention.

### SUMMARY OF THE INVENTION

### Object of the Invention

The object of the invention is to make available a plate or a simple screw fastener system, consisting of at least one locking screw and at least one non-locking screw and a plate, for attachment to bone, with which screws can be screwed in multiaxially with respect to the plate or to the surface of the bone, and the locking screw can be secured against undesired loosening.

### Solution

The underlying concept of the solution is that the plate has at least two through-bores, each through-bore being defined by a midpoint and by a radius. The through-bores themselves are offset relative to each other and intersect each other in such a way that intersection lines are obtained in the direction of the thickness of the plate, these intersection lines interacting with the thread of the locking screw head. A plastic deformation thus takes place between the screw head and/or the intersection lines, and this leads to a force-fit and form-fit connection between plate and locking screw. The non-locking screw can be screwed into a through-bore in the same manner without locking to the plate.

### Advantages of the Invention

One of the main advantages of the invention is that, despite the possibility of multiaxial engagement of the screws, the plate does not need to have a thread. The creation of the through-bore in the plate in itself generates projections or intersection lines, which permit in particular a multi-axial screwing-in of the screws into the plate. The resulting plastic deformation also has the effect that loosening of a locking screw from the plate is possible only with application of considerable force. Unwanted loosening is not possible. While the non-locking screw is securely tightened, it can be loosened with the application of less force than is required to loosen a non-locking screw.

Advantageously, at least three through-bores are provided, which are arranged relative to one another in such a way that all three through-bores intersect. This means that the first through-bore intersects the second through-bore and the third through-bore, the second through-bore intersects the first and third through-bores, and the third through-bore intersects the first and second through-bores. This results in three intersection lines, which extend into the thickness and thus into the depth of the plate. In the middle, a through-opening is centrally obtained whose radius is defined by the distance of an intersection line from the midpoint of the through-opening.

A preferred embodiment provides for a symmetrical configuration of the through-bores starting from a central bore. For this purpose, the through-bores provided in the plate are arranged in such a way that their respective midpoints are arranged on an arc of a circle, starting from the midpoint of the central bore. This produces a flower-shaped, leaf-shaped or cloverleaf-shaped arrangement of the through-bores around the midpoint of the central bore. If several through-bores are used, several projections or intersection lines are also obtained, which can then once again interact with the screw head of the locking screw. The number of through-bores is not limited. The number of through-bores for creating a through-opening is proportional to the intersection lines.

An important advantage is seen in the fact that no defined top face and underside of the bone plate is provided. This means that the plate can be used functionally correctly, irrespective of its position.

All the aforementioned bores of a particular embodiment are cylindrical. This means that the diameter of the through-bores and thus also of the through-opening remains constant over the thickness of the plate.

The intersection lines or intersection surfaces obtained through the cutting of the respective through-bores are configured in such a way that, in a plan view of the plate, they narrow toward the respective center of the through-opening. They have a cross section which is configured in such a way that, starting from the walls of the through-bore, it narrows in the direction of the center of the through-opening. In an illustrative embodiment in which the through-bores are each applied perpendicular to the plate, the cross section, through the depth of the plate, of the respective intersection line or intersection surface is constant. This means that, already when a screw is mounted on the plate according to the invention with the corresponding inventive design of the through-opening, it comes into engagement already with the first turn, so as to permit better screwing in, provision can also be made that the respective through-bore and thus also the whole through-opening is countersunk. In this way, the screw can first be inserted into the through-opening, can be suitably placed there and, only in a further step, can then be brought into engagement with the respective intersection line or intersection surface.

If the through-bores are oblique, that is to say not perpendicular to the longitudinal extent of the plate, this also results in oblique intersection lines or intersection surfaces extending into the depth of the plate. If these are formed at different angles, conical formations are also obtained.

At their free ends, the intersection lines then each have a very small thickness. In this way, it is possible for the thread of a locking screw head to cut in easily in these areas, since the resistance is slight, on account of the small thickness. The V-shaped configuration of the areas of the intersection lines, as seen in plan view, are such that the cross section becomes thicker the greater the distance from the free end of the intersection line. This is particularly so when the locking screw heads are of conical shape, a greater cross section being penetrated by the thread of the locking screw head as the screwing-in of the screw head increases. A strong plastic deformation thus takes place. The non-locking screw head can be conically shaped, cylindrical or partially spherical and it is sized to fit in the through-bores.

In a preferred embodiment, after production of a central bore, the through-bores are not formed cylindrically in the direction of thickness of the plate, but conically. The conicity is such that the through-bore narrows from the screwing-in direction toward the bone. This is advantageously achieved through using a conical milling cutter. Its axis of symmetry is preferably parallel to the axis of the central bore and also perpendicular to the plate. The midpoints of the through-bores are chosen such that their radius, which is preferably the same in all the through-bores, intersects the diameter of the central bore, resulting in a through-opening which is larger than the diameter of the central bore and, on its outer wall, has a large number of conical walls. The individual walls are conically shaped and narrow in the screwing-in direction. Thus, in the screwing-in direction, intersection lines are first obtained (since the diameter of the individual through-bores is large), whereas with increasing depth (in the screwing-in direction) the diameter of the through-bores decreases, because of the provided conicity, and intersection surfaces are therefore obtained between the individual through-bores.

It was found that the number of through-bores is almost proportional to the screwing-in force of the screw. This means that, with a larger number of through-bores (for example 15), the screwing-in force is smaller compared to a smaller number of through-bores (for example 5).

Provision is made in principle for more force to be applied as the depth of insertion of a screw into the plate increases. The reason for this is that first an interaction takes place, preferably a deformation with the intersection lines. The line or volume provided in engagement with the screw is small. When the intersection line merges into an intersection surface, the volume to be deformed by the screw becomes greater, and, consequently, more force has to be applied. However, this also has the effect of achieving a secure, non-releasable connection between the plate and the screw when a locking screw is used.

In a preferred development, the central bore is first produced with a conical cross section. A defined conicity is chosen. The through-bores, which follow in the shape of a cloverleaf in relation to this central bore, also have a conicity, but a different one than the central bore. In this way, intersection surfaces, not intersection lines, are obtained in the remote screwing-in area within the through-bore.

Screw heads and screw shanks preferably have the same diameter. This affords the advantage that the screw is easy to produce.

Alternatively, provision can also be made for the screw head to be made cylindrical and, in the case of locking screws, to have a different thread turn than the rest of the shank.

In another alternative, the screw head can be made conical and thus adapts to the shape of the through-bore.

The screw shank itself can also be made conical, in order thereby to reduce the force applied, particularly when screwing the screw into the bone.

In another alternative embodiment, the screw head is made at least partially spherical. In this way, it is possible to achieve a greater angle, deviating from the line perpendicular to the plate, by the screw still being able to be screwed obliquely (multiaxially) in the plate.

Thus, a system has been proposed consisting exclusively of three structural parts. It is not necessary to use additional expanding elements or shim elements or other materials. Screws and plate can preferably be made from the same material. To further improve the plastic deformation, particularly in the area of the projections, provision can be made for the plate to be produced from a softer material than the screw itself.

One of the main advantages of the system lies in the fact that the plate itself can be produced with minimal outlay. The simplest form is to provide three through-bores, which are to be configured in a defined arrangement to one another, such that a through-opening is obtained into which the screws can be inserted.

In the preferred embodiment, a central bore is to be provided in the plate, which is cut from through-bores, said through-bores being produced with a conical mill, which narrows in the screwing-in depth. The number of through-bores should be greater than 5. A preferred number is 15.

In another advantageous embodiment, the intersection lines and/or intersection surfaces are hardened and thus provide a harder property compared to the rest of the material of the plate. Such hardening can be carried out by laser, for example.

Other advantageous embodiments will become clear from the attached drawings, from the description, and also from the claims.

### BRIEF DESCRIPTION OF THE DRAWINGS

FIG. 1 shows a first illustrative embodiment of a schematic representation of a through-opening of a plate as part of the screw fastener system according to the invention;

FIG. 2 shows a second illustrative embodiment of a schematic representation of a through-opening of a plate as part of the screw fastener system according to the invention;

FIG. 3 shows a third illustrative embodiment of a schematic representation of a plate as part of the screw fastener system according to the invention;

FIG. 4A shows a schematic representation of the orthopedic screw fastener system in cross section, a locking screw having already been screwed into the plate and bone;

FIG. 4B shows a schematic representation of the orthopedic screw fastener system in cross section, a non-locking screw having already been screwed into the plate and bone;

FIG. 5A shows a perspective view of the screw fastener system according to FIG. 4A;

FIG. 5B shows a perspective view of the plate without screws according to FIG. 5A;

FIG. 6 shows a perspective view of a plate with a multiplicity of through-openings, one through-opening being provided with a screw;

FIG. 7 shows a schematic representation of a through-opening located in a plate and consisting of several cylindrical through-bores, to explain the production;

FIG. 8 shows another schematic representation of a through-opening located in a plate and consisting of several conical through-bores;

FIG. 9 shows another schematic representation of another illustrative embodiment of a through-opening located in a plate and consisting of several conical through-bores.

### DETAILED DESCRIPTION OF A PREFERRED EMBODIMENT

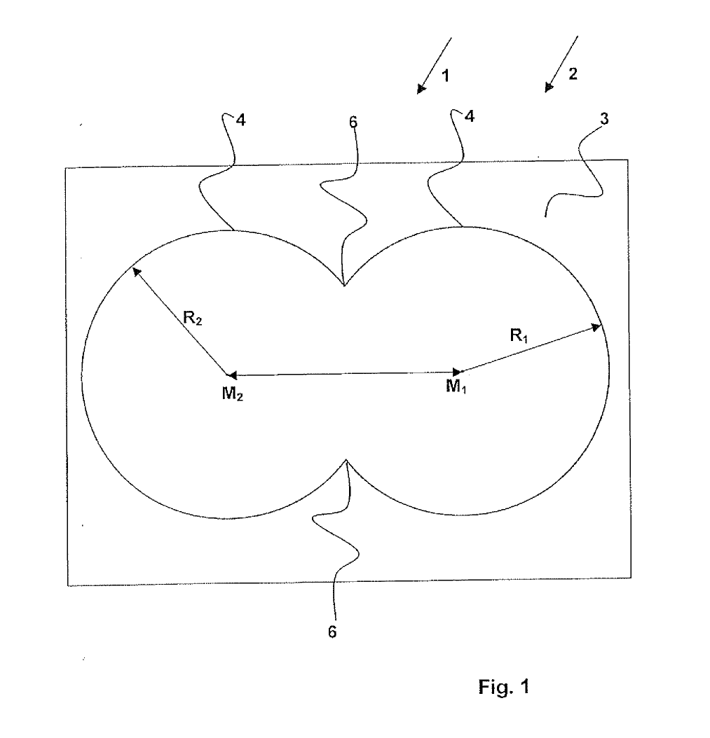

FIGS. 1 to 3 show a selection of possible through-openings 1 for an orthopedic screw fastener system 2. To produce such a through-opening 1 according to the invention, arranged on a plate 3, at least two through-bores 4 are generated. Each through-bore 4, is defined by a radius Rₓ and a midpoint Mₓ

According to a first illustrative embodiment, as shown in FIG. 1, the through-opening 1 consists of two through-bores 4 whose two midpoints M₁, M₂ are arranged at a distance 5 from each other. The distance 5 is to be chosen such that the two radii R₁, R₂ of the through-bores 4 intersect. This results in intersection lines 6, which extend into the depth (thickness) of the plate 3. The intersection lines 6 are configured in such a way that they are designed tapering toward the free space of the through-opening 1 and have a thicker wall thickness as their distance from the latter increases. In this way, it is possible that the free ends thus formed can be plastically deformed by screwing-in of a screw.

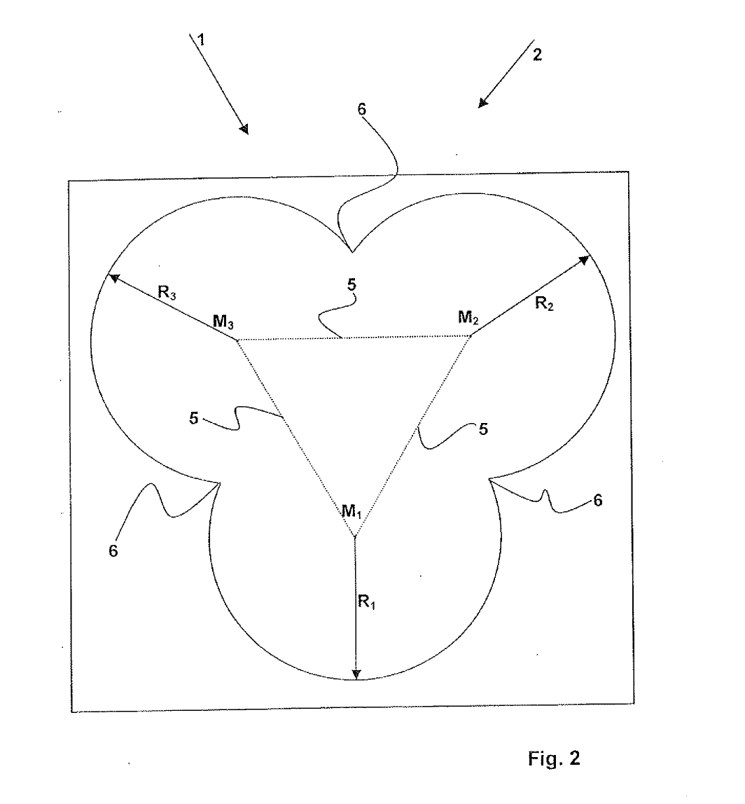

According to another illustrative embodiment, as shown in FIG. 2, the through-opening 1 consists of three through-bores 4 whose midpoints M₁, M₂, M₃ are each arranged at a distance 5 from one another. The respective distance 5 is to be chosen such that the three radii R₁, R₂, R₃ of the through-bores 4 intersect. This results in intersection lines 6, which extend into the depth (thickness) of the plate 3.

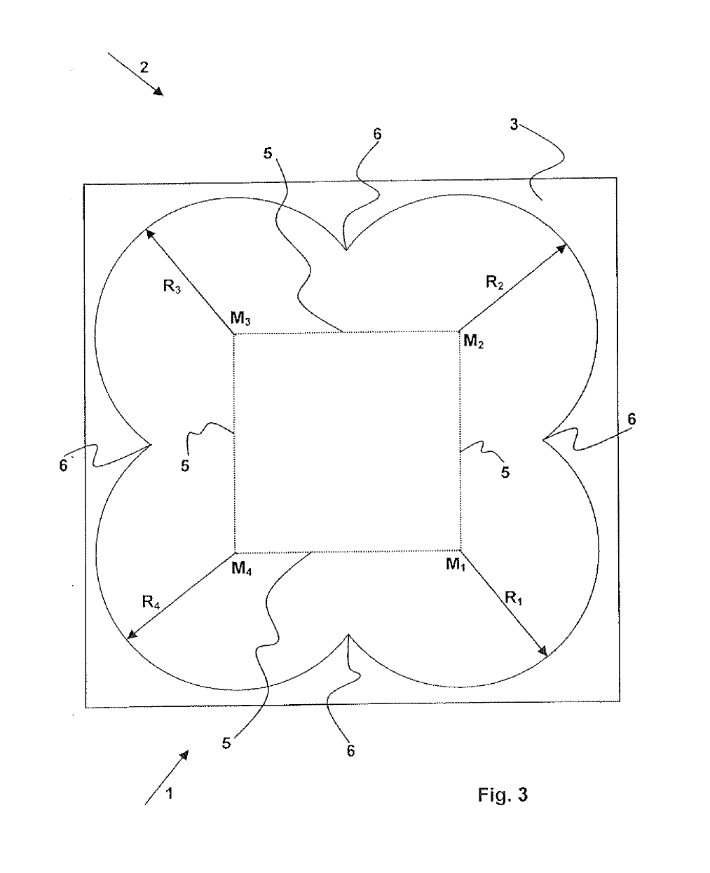

According to another illustrative embodiment, as shown in FIG. 3, the through-opening 1 consists of four through-bores 4 whose midpoints M₁, M₂, M₃ M₄ are each arranged at a distance 5 from one another. The respective distance 5 is to be chosen such that the four radii R₁, R₂, R₃, R₄ of the through-bores 4 intersect. This results in intersection lines 6, which extend into the depth (thickness) of the plate 3.

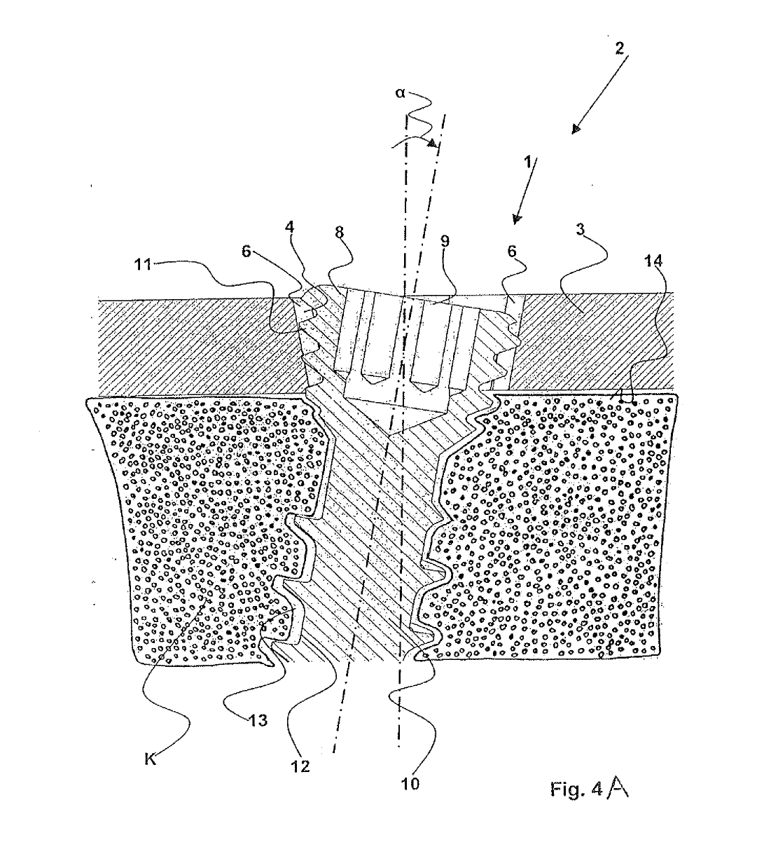

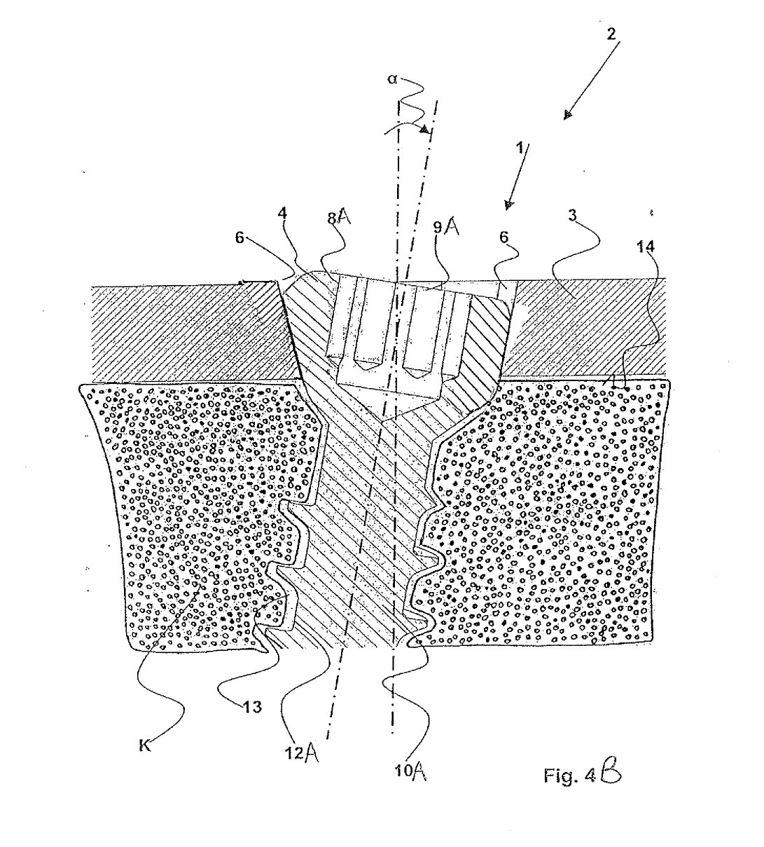

FIGS. 4A, 4B, 5A, 5B and 6 show the orthopedic screw fastener system 2. It consists of a locking screw 8 and a non-locking screw 8A and of the plate 3, as is shown for example in FIGS. 1 to 3. The plate 3 is used for fixing a fracture, for example. The locking screw 8 itself is screwed into a bone K. The plate 3 lies with its underside 14 on the outer face of the bone K (FIGS. 4A and 4B). The through-opening 1, as is shown by way of example in FIGS. 1-3, is used to receive the locking screw 8, which consists of a screw head 9 and of a screw shank 10. The screw head 9 and the screw shank 10 each have a thread. The thread 11 of the screw head 8 interacts with the intersection lines 6. The thread 12 of the screw shank 10 interacts with the bone K. In the bone K itself, a bore 13 is preferably already provided whose clear width is smaller than the diameter of the screw shank 10. By virtue of the fact that the clear width of the through-opening 1 is greater than that of the diameter of the screw head 9, it is possible, as is also shown in FIGS. 4A and 4B, to arrange the locking screw 8 and the non-locking screw 8A not perpendicular to the plate 3, but inclined at any desired angle. Non-locking screw 8A has a head 9A, a screw shank 10A and a thread 12A on the screw shank. The non-locking screw 8A is screwed into the through-opening 1 and bone K in the same manner as the locking screw 8.

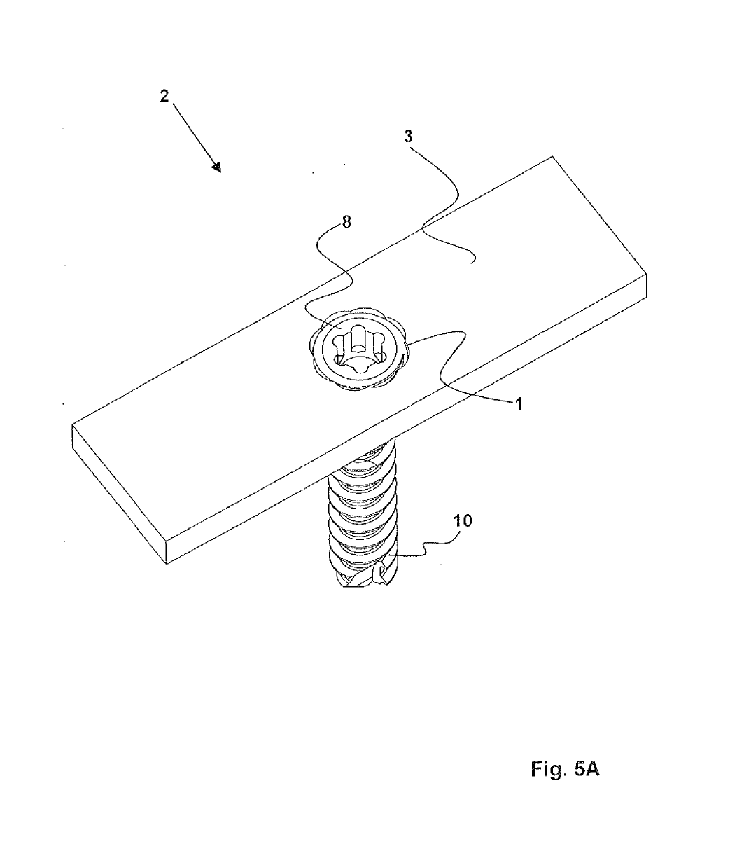

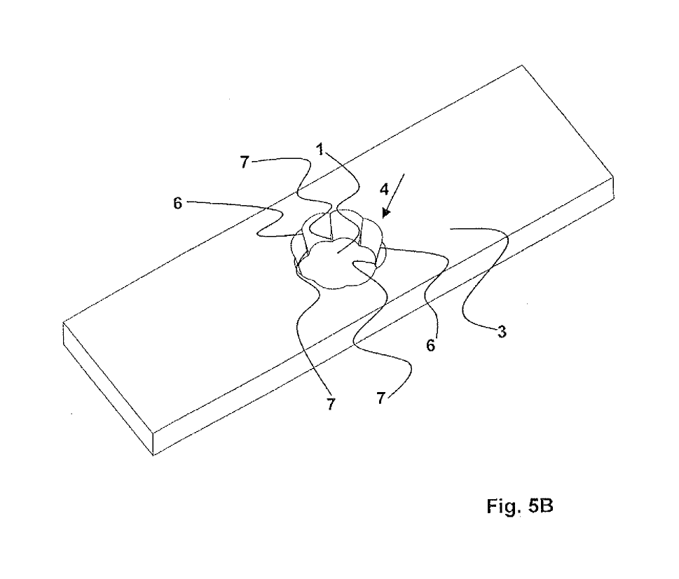

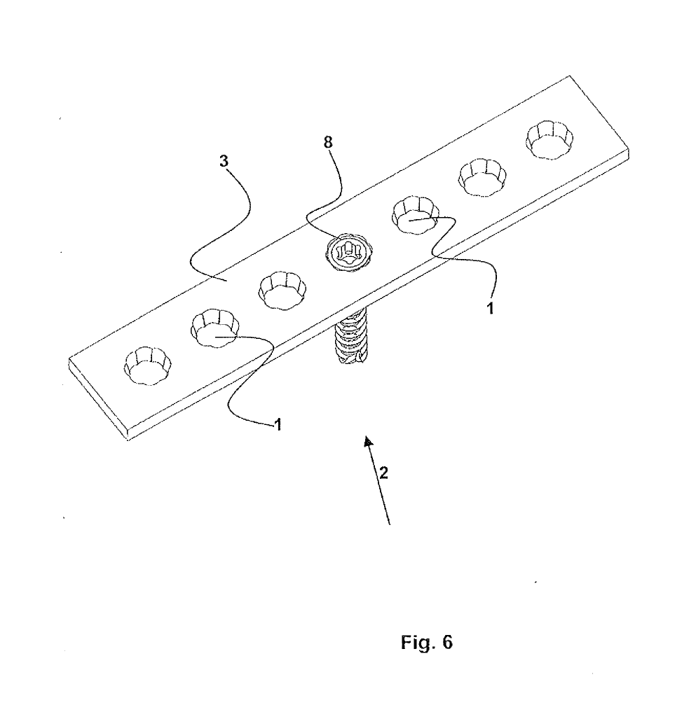

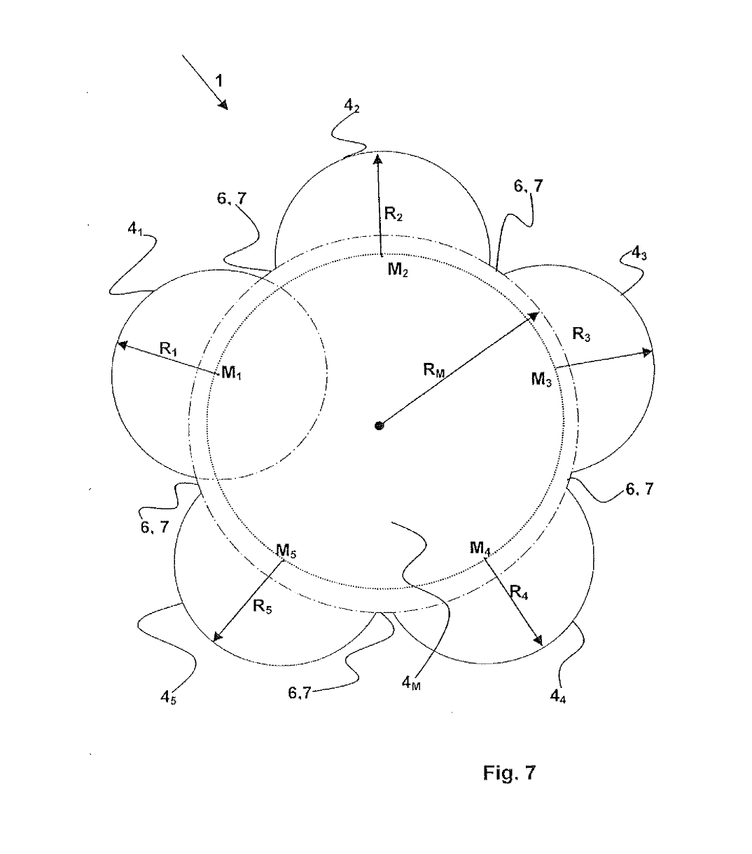

FIG. 7 is a schematic representation of how a through-opening 1, as shown in FIG. 5B and FIG. 6, is constructed. First, the midpoint M of the through-opening 1 is determined. A radius RM is then determined. This serves to ensure that all the other midpoints M₁, M₂, M₃ M₄, M₅ lying on the circle with the radius RM are at the same distance from the midpoint R_{M}. The circle with the midpoint R_{M} is then divided up in such a way that any desired number of other midpoints for other circles are arranged at the same distance from one another. Here in FIG. 6, the circle is divided up in such a way that the midpoints M₁, M₂, M₃ M₄ M₅ are at the same distance from one another.

A central bore 4_{M} with midpoint M and radius R_{M} is first to be generated. The central bore 4M, in the illustrative embodiment shown, is cylindrical and uniform through the whole thickness of the plate 3. The first through-bore 4₁ with a midpoint M₁ and a radius R₁ is then generated. As soon as the bore is formed, a cylindrical through-bore 4₁ is obtained. At the points M₂ to M₅, a through-bore 4₂ . . . 4₅ is then formed, specifically with radii R₂ . . . R₅, where the radii R₁ . . . R₅, are identical. This gives rise to the cloverleaf shape with the respective intersection lines 6, which interact with the screw head 9. This embodiment affords the possibility, after production of a central bore 4M, of providing a multiplicity of through-bores 4ₓ with radius Rₓ and midpoint Mₓ. Here, x is an element of the natural numbers from 1 to infinity.

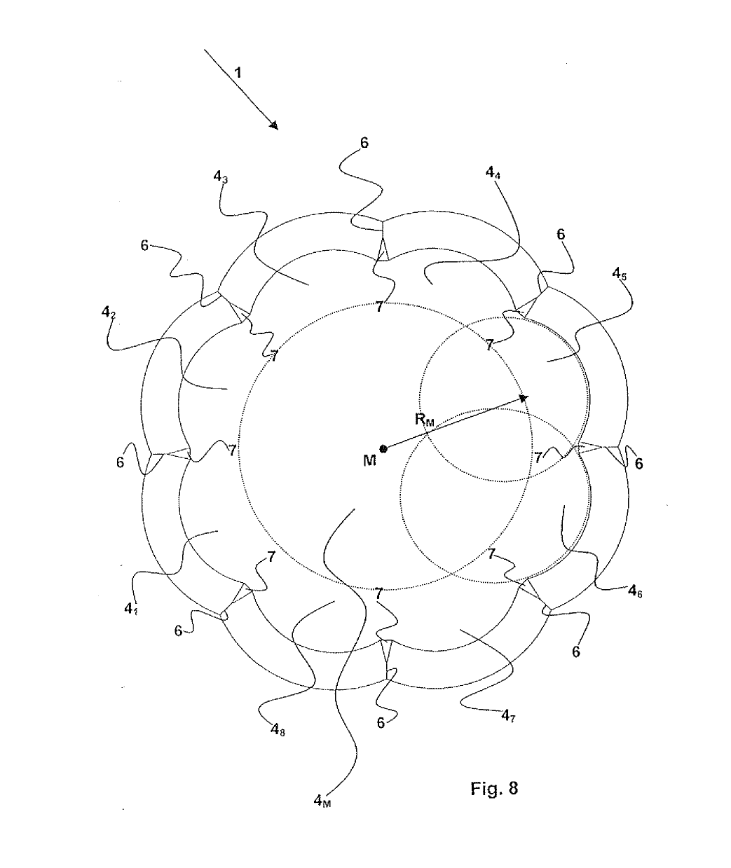

FIG. 8 shows another embodiment of the configuration according to FIG. 7. First, a central bore 4_{M} with midpoint M and radius R_{M} is to be generated. The central bore 4_{M} in the illustrative embodiment shown, is conical in shape and narrows through the thickness of the plate 3, starting from the top face of the plate. The defined conicity, that is to say a change of the diameter of the cone as a function of the thickness of the plate, is chosen such that the diameter narrows in the screwing direction of the screw. By virtue of the fact that the other through-bores fully intersect the central bore 4_{M} it suffices for the central bore 4_{M} to be designed cylindrically.

The first through-bore 4₁ with a midpoint M₁ and a radius R₁ is then generated. The through-bore 4₁, in the illustrative embodiment shown, is not conical in shape narrows through the thickness of the plate 3. As soon as the bore is formed, a conical through-bore 4₁ is obtained. At the points M₂ to M₅, a through-bore 4₂ . . . 4₅ is then once again formed, specifically with radii R₂ . . . R₅ where the radii R₁ . . . R₅ and the conicities are identical. This gives rise, in a plan view, to the cloverleaf shape with the respective intersection lines 6, which interact with the screw head 9. Additional intersection surfaces 7 are additionally obtained on account of the conicities of the through-bores, preferably in continuation of the intersection line 6. This means that the intersection lines 6 merge with increasing depth into intersection surfaces 7. In FIG. 8 is shown the upper radius R₆ₒ of the through-bore 46 with the midpoint M₆. The lower radius R₆ₒ is also shown. A milling-cutter is using for producing the mentioned through-bores 4ₓ. This milling-cutter shows these to radii Rₓᵤ and Rₓₒ. The radii R₅ₒ, R₅ᵤ, R₆ₒ, R₆ᵤ are only shown for illustrating the manufacturing of the though-opening 1. Radii Rₓᵤ, Rₓₒ as well the midpoints Mₓ have to be transformed to each through-bore 4ₓ.

After using said milling-cutter for making the through-bores 4, seen from the top the intersection lines 6, the intersection surfaces 7 and the surfaces F on the top of the plate are formed. In the illustrative embodiments shown in FIGS. 5B and 8, the intersection surfaces 7 are on that side of the plate 3 facing toward the bone and occupy about ⅓ of the depth of the plate.

The number of through-bores can be chosen freely. It has been found that a multiplicity of through-bores reduces the screwing-in force of the screw. The more through-bores there are, the greater the number of intersection surfaces and intersection lines. However, the present thickness (seen from the plan view) of the intersection lines and surfaces is smaller.

In contrast to FIG. 8, more through-bores are provided, and the through-bores 4₁ . . . 4₈ are conical, these through-bores narrowing toward the bone K.

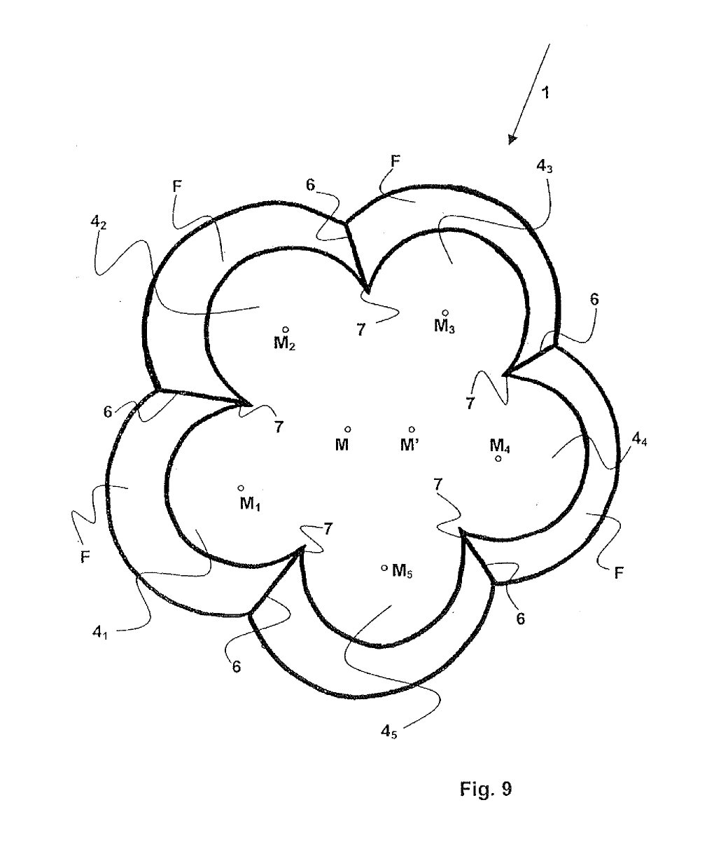

FIG. 9 shows another embodiment where the conical surfaces, in contrast to FIG. 8, are of asymmetrical design. The asymmetry is obtained by providing two central through-bores, each with a midpoint M, M′. Starting from these respective midpoints, there extend in different distance the respective midpoint Mₓ (M₁ . . . M₅) in different length or different distance. This gives rise, in plan view, to a rosette-shaped configuration of the through-opening 1 according to the invention, a slightly offset rosette formation which forms oblique bearing faces F of different length. The intersection lines 6 seen in plan view then merge, as also in the previous examples, with increasing depth into intersection surfaces.

FIGS. 5A to 9 in particular show embodiments of the through-opening according to the invention of special configuration. As a result of the conical design of the respective through-opening, an intersection line is first obtained in the screwing direction which, with increasing depth in the direction of the bone, forms an intersection surface. This can be clearly seen from FIG. 5B in particular. This affords the advantage that only a few thread windings initially interact with the intersection line during screwing-in and exert there a slight plastic deformation, and the deeper the locking screw is screwed in the greater the interaction between the intersection line or intersection surface and the thread of the respective locking screw has to be. This has the result that, because of the plastic deformation, a connection is obtained that is almost non-releasable.

As an alternative to this conical design, provision is also made that the through-bores arranged around the central bore are each arranged at an angle, such that the through-opening also narrows toward the bottom. In this way, it is not necessary to produce conical bores exclusively. It is therefore also possible, after the central bore has been produced, to provide such configurations as are shown in FIGS. 5 to 9. By the oblique arrangement of the respective drills for producing the through-bore with respective midpoint Mx and radius Rx, where x is an element of the natural numbers 1 to infinity, it is therefore possible that a self-locking screw fastener system formed of locking screw and plate be used in the orthopedic sector.

The orthopedic screw fastener system thus represents an important innovation over the prior art, since it consists exclusively of three elements, but has properties hitherto known only with several elements in a complex construction. As a result of the cloverleaf-shaped structure of the through-opening, the screws can be screwed in at different angles. The system is distinguished by the fact that the locking screw is secured against undesired loosening while the non-locking screw can be intentionally loosened more easily than the locking screw.

What is claimed is:

1. An orthopedic screw fastener system, comprising
at least one locking screw, comprising in each case a screw head and a screw shank, the screw head being provided with a thread,
at least one non-locking screw, comprising in each case a screw head and a screw shank, the screw head being non-threaded,
and
at least one plate, which has a suitable thickness, provided with at least two through-bores which are able to receive the screws,
wherein
the plate has at least two through-openings, each formed by at least two through-bores, each through-bore being defined by a midpoint and by a radius,
the through-bores are offset relative to each other and intersect each other in such a way that intersection lines and/or intersection surfaces extend into the depth of the through-opening.

2. The screw fastener system as claimed in claim 1, wherein the intersection surfaces and/or intersection surfaces extend in the longitudinal direction of the through-bores.

3. The screw fastener system as claimed in claim 1, wherein the midpoints of the through-bores are arranged on a common arc of a circle.

4. The screw fastener system as claimed in claim 1, wherein, in the case of three or more through-bores, these are offset relative to on another in such a way that each through-bore intersects all the other through-bores at least once.

5. The screw fastener system as claimed in claim 1, wherein at least one through-bore has a conical shape.

6. The screw fastener system as claimed in claim 5, wherein the through-bores narrow in the screwing-in direction.

7. The screw fastener system as claimed in claim 5, wherein, in the screwing-in direction of the through-opening, intersection lines are first provided, which merge into intersection surfaces.

8. The screw fastener system as claimed in claim 1, wherein a through-opening can be produced by producing a central bore with a radius about a midpoint M, and subsequent further through-bores that are arranged like a cloverleaf around the central bore.

9. The screw fastener system as claimed in claim 1, wherein the screw head is conical and narrows toward the screw shank.
